(19) United States
(12) Patent Application Publication
(10) Pub. No.: US 2012/0202043 A1
(43) Pub. Date: Aug. 9, 2012

(54) Antimicrobial Composite Structure

(76) Inventors: Nathan G. Bonn-Savage, Portland, OR (US); Jon N. Neese, Oregon City, OR (US)

(21) Appl. No.: 13/351,744

(22) Filed: Jan. 17, 2012

Related U.S. Application Data
(60) Provisional application No. 61/433,647, filed on Jan. 18, 2011.

(51) Int. Cl. B32B 3/26; B82Y 5/00; B82Y 30/00

(52) U.S. Cl. 428/315.5; 977/773; 977/810; 977/904; 977/788; 977/931

(57) ABSTRACT

An antimicrobial composite structure including: a polymeric material, the material having a first exterior surface expanded to fibrils and nodes, a second exterior surface and an interior portion between the exterior surfaces; and nanoparticles present in the interior portion adjacent the first exterior surface but not in the interior portion adjacent the second exterior surface. The nanoparticles can be silver nanoparticles and the polymeric material may be an expanded fluoropolymer material such as expanded polytetrafluoroethylene.

This application claims the benefit of priority from U.S. Provisional Application No. 61/433,647 filed on Jan. 18, 2011, and U.S. patent application Ser. No. 13/336,193 filed on Dec. 23, 2011.

### FIELD OF THE INVENTION

The invention relates to medical articles that include an antimicrobial coating. More particularly, the invention relates a medical device, device surface, or material surface having an applied silver nanoparticle coating.

### BACKGROUND OF THE INVENTION

Application of antimicrobial agents such as metal nanoparticles or antibiotic coatings to surfaces such as, for example, surfaces of medical devices or other material surfaces are typically conducted in a batch style process due to difficulty in maintaining reagent stability and coating uniformity in continuous processes. Exemplary batch style processes may include vapor deposition, direct incorporation of the antimicrobial agent in a material forming the surface, dipping of the device into a bath containing the active agent and a binder material, or a combination of the above processes. Existing methods typically cannot be adapted to continuous or in-line processes and can include the incorporation of expensive equipment, operator skill, and labor intensive steps, Also certain substrates provide a particular challenge in that they require selective application on detailed geometries or are porous and have a requirement that the application be limited as to the depth of impregnation. Currently available dipping processes for the application of coating agents are difficult to implement and generally provide coatings of insufficient concentration tolerances for the desired application herein.

A typical dip type coating can apply silver, Ag, to the surface of a material, but the process is relatively uncontrolled and variable. An example illustrating the variability of results from a dip coating process is shown in FIG. 1 which is a graph of silver deposition expressed in units of micrograms per square centimeter on the y-axis and the number of dips on the x-axis. More particularly, the item dipped was an expanded polytetrafluoroethylene (ePTFE) vascular graft. The graft was deposited in a liquid bath containing a silver nanoparticle and heptane mixture. Each dip or immersion of the article was timed to last for 30 seconds. The sample was air-dried for 5 minutes between dips. The silver deposition was measured utilizing flame atomic absorption spectrophotometry (FAAS).

As is evident from FIG. 1, the number of dips did not correlate well with a predictable or generally uniform increase in the density of silver on the surface.

Accordingly, there is a need for a coating process that can be tightly controlled to provide a relatively predictable and uniform deposition of a metal nanoparticle such as silver nanoparticle. There is also a need for a process that allows selective application of an antimicrobial nanoparticle, flexibility of delivery vehicle (meaning a variety of organic solvents can be employed depending on substrate material), and coating concentration. Moreover, there is a need for silver-containing, non-aqueous formulations that can be the basis of a coating process that is flexible and provides a controllable and relatively predictable and uniform deposition of silver nanoparticles. This need extends to materials that can be produced by such a coating process. There is a need for a fluoropolymer material having satisfactory antimicrobial properties. This need is specifically apparent because of the difficulty of applying coatings to fluoropolymer materials.

### SUMMARY OF THE INVENTION

The present invention addresses the problems described above by providing an antimicrobial composite structure that includes: a microporous polymeric material, the material having a first exterior surface, a second exterior surface and an interior portion between the exterior surfaces; and nanoparticles present in interstices adjacent the first exterior surface but not in interstices adjacent the second exterior surface.

According to the invention, the microporous polymeric material may be a matrix of expanded polymer material. For example, the first exterior surface may be an expanded fluoropolymer material and may have nodes and fibrils of expanded fluoropolymer material. Desirably, the expanded fluoropolymer material may be expanded polytetrafluoroethylene.

The nanoparticles may be metal nanoparticles and desirably are silver nanoparticles. The composite structure may further include copper nanoparticles, chlorohexidine, iodine, antibiotics and combinations thereof. According to an aspect of the invention, the nanoparticles are present in the microporous polymeric material beginning at or preferably adjacent the exterior surface and are distributed into the microporous polymeric material to a predetermined depth. For example, the nanoparticles may be present in interstices of the microporous polymeric material up to a depth of about 100 micrometers. As another example, the nanoparticles may be present in interstices of the microporous polymeric material up to a depth of about 50 micrometers. As yet another example, the nanoparticles may be present in interstices of the microporous polymeric material up to a depth of about 25 micrometers. According to the invention, the nanoparticles may be present in interstices of the microporous polymeric material up to a depth of from about 5 to about 20 micrometers. For example, the nanoparticles may be present in a matrix of expanded fluoropolymer material such as expanded polytetrafluoroethylene up to a depth of about 50 micrometers. As another example, the nanoparticles may be present in a fluoropolymer material such as polytetrafluoroethylene expanded to a matrix of nodes and fibrils up to a depth of about 50 micrometers.

In an aspect of the invention, the distribution of nanoparticles into the interstices of the polymeric material (e.g., into the matrix of expanded polymeric material) to a predetermined depth help make the nanoparticles resist removal by frictional forces applied to the exterior surface of the matrix. For example, the distributing nanoparticles into the matrix to a predetermined depth help make the nanoparticles resist removal wiping.

In yet another aspect of the invention, the antimicrobial composite structure may form an area, region, portion, or dimension of a medical device, device material, packaging material or combinations thereof.

According to an aspect of the invention, such antimicrobial composite structures are produced by a process for depositing nanoparticles on a surface such as a matrix of expanded fluoropolymer material such that the nanoparticles penetrate the matrix. The process involves providing a sol composed of a volatile non-aqueous liquid and nanoparticles suspended in the non-aqueous liquid. The sol may be provided by preparing an aqueous suspension of nanoparticles and extracting the nanoparticles into a non-aqueous liquid to form a sol. For example, the sol may be prepared by forming an aqueous suspension of silver nanoparticles and extracting the silver nanoparticles into a non-aqueous liquid. Any water immiscible organic solvent may be used in the extraction process.

The sol desirably has low viscosity and is adapted to forming droplets utilizing conventional droplet forming techniques. The sol is then processed to form a plurality of droplets. These droplets are deposited on a surface of the matrix and penetrate into the matrix. Finally, the non-aqueous liquid is evaporated from the surface to leave a residue of nanoparticles. Alternatively and/or additionally to forming droplets, it is contemplated that the process may deposit the sol on a surface of the matrix by techniques selected from printing, dipping, brushing or combinations thereof.

Generally speaking, the volatile non-aqueous liquid component of the sol may be any water immiscible organic solvent that has a sufficiently low viscosity for an application process such as spraying has a high volatility to be quickly evaporated, is compatible with the nanoparticles, can be readily handled in an application process, and has surface energy that allows the sol to penetrate into a matrix of expanded fluoropolymer material. For example, the liquid may be selected from benzene, butanol, carbon tetrachloride, cyclohexane, 1,2-dichloroethane, dichloromethane, ethyl acetate, ethyl ether, iso-octane, methyl-t-butylether, methyl ethyl ketone, pentane, heptane, chloroform, toluene, and hexane and mixtures thereof. Desirably, the nanoparticle component of the sol is silver nanoparticles. The silver nanoparticles may have an effective diameter of less than 20 nanometers (nm). Even more desirably, the residue of nanoparticles (i.e., the nanoparticles deposited into the matrix of expanded fluoropolymer material) provides antimicrobial properties. It is contemplated that the sol may further include other materials having antimicrobial properties including, but not limited to, copper nanoparticles, chlorohexidine, iodine, antibiotics and combinations thereof.

The plurality of droplets may be formed by a spray process. For example, the spray process may utilize a centrifugal pressure nozzle, a solid cone nozzle, a fan spray nozzle, a sonic atomizer, a rotary atomizer, a flashing liquid jet, ultrasonic nozzles or combinations thereof. The spray process may utilize electrostatic charge. The expanded fluoropolymer material to be treated may be a particular area, region, portion, or dimension of a medical device, device material, packaging material or combinations thereof.

In an aspect of the invention, the steps of depositing the plurality of droplets on a surface and evaporating the non-aqueous liquid from the surface leaving a residue of nanoparticles in the expanded matrix of fluoropolymer material may be conducted a plurality of times. That is, the process may deposit nanoparticles on a porous surface such as an expanded matrix of fluoropolymer material that the nanoparticles penetrate the porous surface or matrix. More particularly, the process may deposit nanoparticles on a porous surface such as an expanded matrix of fluoropolymer material in such manner that the penetration of nanoparticles into the porous surface or matrix is controlled.

The present invention also encompasses an article including a surface such as an expanded matrix of fluoropolymer material containing nanoparticles deposited according to any of the above-described processes or system. Desirably, the article surface is a matrix having a first exterior surface, a second exterior surface and an interior portion between the exterior surfaces; and the nanoparticles are present in the interior portion adjacent the first exterior surface but not in the interior portion adjacent the second exterior surface.

Other objects, advantages and applications of the present disclosure will be made clear by the following detailed description.

### DESCRIPTION OF THE DRAWINGS

FIG. 1 is an illustration of a graph of silver deposition provided by a conventional dip process. The silver deposition is expressed in units of micrograms per square centimeter on the y-axis and the number of dips on the x-axis.

FIG. 2 is a schematic view illustration showing an exemplary apparatus used in a process for deposition of nanoparticles.

FIG. 3A is a left side view illustration showing an exemplary spray head of an exemplary apparatus shown in FIG. 2 used in a process for deposition of nanoparticles.

FIG. 3B is a front view illustration showing an exemplary spray head of an exemplary apparatus shown in FIG. 2 used in a process for deposition of nanoparticles.

FIG. 3C is a top view illustration showing an exemplary spray head of an exemplary apparatus shown in FIG. 2 used in a process for deposition of nanoparticles.

FIG. 4 is an illustration of a graph of silver deposition provided by an exemplary process for deposition of nanoparticles as illustrated in FIGS. 2 and 3. The silver deposition is expressed in units of micrograms per square centimeter on the y-axis and the number of spray passes on the x-axis.

FIG. 5 is an optical image of a cross-section of an exemplary antimicrobial composite material in the form of a treated tube of expanded polytetrafluoroethylene.

FIG. 6 is a scanning electron microphotograph of a portion of an exemplary antimicrobial polymeric material in the form of an expanded polymer matrix at a linear magnification of 200×.

FIG. 7 is a scanning electron photomicrograph of a portion of an exemplary antimicrobial polymeric material in the form of an expanded polymer matrix showing a portion of the expanded polymer matrix of FIG. 6 at a linear magnification of 2000×.

FIG. 8 is an illustration of a graph of the amount of silver nanoparticles from an exemplary antimicrobial composite material. The amount of detected silver is expressed as a weight percentage on the y-axis and the relative position of the test sample on an article is on the x-axis.

FIG. 9 is a photograph (backlit) of an exemplary sheet highlighting the treated portion is approximately 28.5 cm×9.6 cm in size. The film was sprayed horizontally with the silver nanoparticle sol along the long axis of the film. Three zones were created by varying the number a treatment passes: 15 passes (top); 25 passes (middle); and 30 passes (bottom).

FIG. 10 is an illustration of a graph of the amount of silver nanoparticles from a comparative polytetrafluoroethylene film material and from an antimicrobial composite material. The amount of detected silver is expressed as a weight percentage on the y-axis and the number of treatment passes of the comparative film material is on the x-axis, except for the antimicrobial composite material—which illustrates a summary of the range illustrated in FIG. 8.

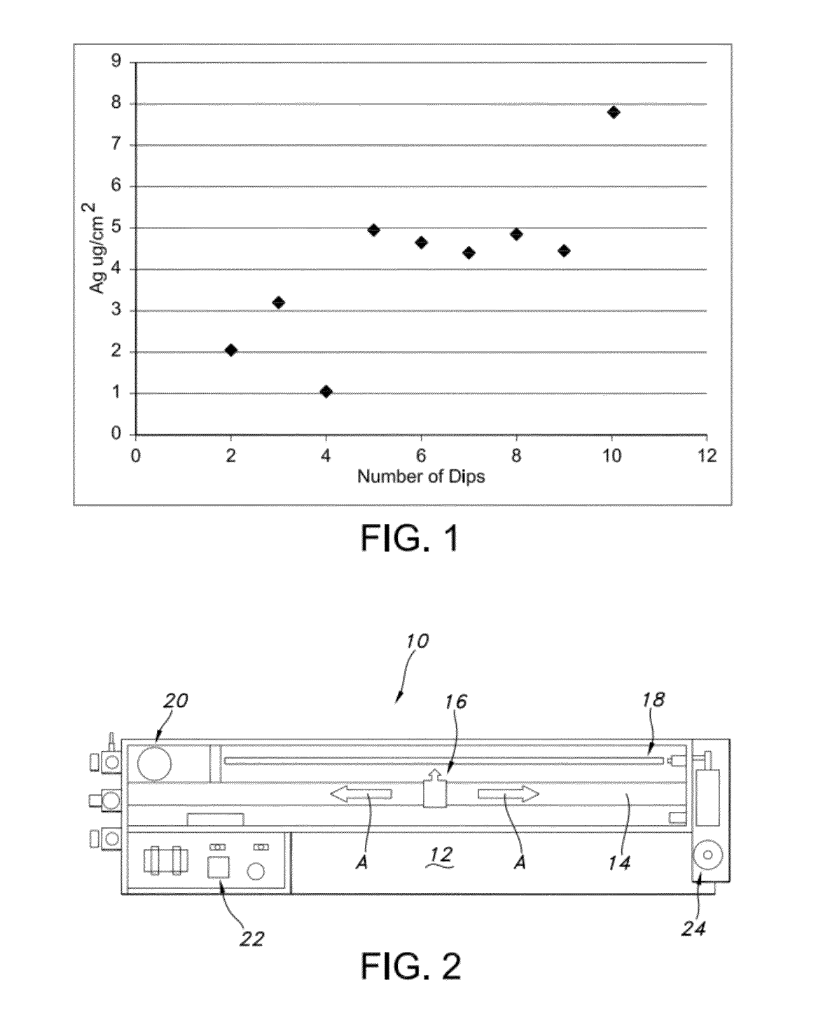

### DETAILED DESCRIPTION

To illustrate the invention and demonstrate its operation, various articles were prepared by applying silver nanoparticles (occasionally referred to herein as “nanosilver”) onto selective surfaces of various materials including microporous polymeric materials such as, for example, matrices of expanded polymer material. However, it is contemplated that the metal nanoparticle may be gold, platinum, indium, rhodium, palladium, copper or zinc. The nanoparticles may be in the size range of 0.1 to 100 nm. These nanoparticles may have a standard normal size distribution; however, nanoparticles less than about 20 nm have been found to work well.

The silver nanoparticles were applied or deposited onto surfaces of expanded fluoropolymer materials from a sol composed of a volatile non-aqueous liquid and nanoparticles suspended in the non-aqueous liquid. The sol may be readily provided by preparing an aqueous suspension of nanoparticles and extracting the nanoparticles into a non-aqueous liquid to form a sol. Suitable techniques may be found at, for example, U.S. Patent Application Publication No. 2007/0003603 for “Antimicrobial Silver Composition” published Jan. 4, 2007, the contents of which are incorporated herein by reference.

Generally speaking, the liquid component of the sol is any volatile water immiscible organic solvent that has a sufficiently low viscosity for the application process (e.g., spraying), has a relatively high volatility to be quickly evaporated, is compatible with the nanoparticles, and can be readily handled in an application process. For example, the liquid may be selected from benzene, butanol, carbon tetrachloride, cyclohexane, 1,2-dichloroethane, dichloromethane, ethyl acetate, ethyl ether, iso-octane, methyl-t-butylether, methyl ethyl ketone, pentane, heptane, chloroform, toluene, and hexane and mixtures thereof. Silver nanoparticles having an effective diameter of less than 20 nm have been found to work well. A silver nanoparticle sol having a viscosity of about 1 cP or less at 25° C. has been found to work well. The viscosity of the nanoparticle sol at the typical concentrations of nanoparticles (e.g., 25 to 5000 parts per million) will have a viscosity of the volatile water immiscible organic solvent. Of course, the viscosity may be determined utilizing viscometers such as a Brookfield RV DV-E Viscometer with Helipath Spindle Set (T-bar spindles). However, the viscosity may be so low that it may be only possible to determine that the viscosity is less than 1 cP with conventional viscometers.

The surface to be treated is desirably a microporous polymeric material. While various microporous polymeric materials are contemplated, it is desirable that the polymeric materials have a relatively open structure that allows nanoparticles to penetrate into to portions or areas or interstices in the material immediately adjacent the surface such that the nanoparticles are not present or exposed on top of the surface like on the surface of smooth film but are instead present predominately below the outermost surface.

For example, a microporous polymeric material may be in the form of an expanded polymeric material such as an expanded fluoropolymer (e.g., polytetrafluoroethylene), expanded polyester (e.g., polyethylene terephthalate), expanded polyethylene (e.g., ultra high molecular weight polyethylene) or the like. The expanded polymer material desirably is in the form of a porous matrix or porous structure that may be described as a node and fibril microstructure. Such microstructure is described at, for example U.S. Pat. No. 3,962,153 for Very Highly Stretched Polytetrafluoroethylene and Process Therefore issued Jun. 8, 1976 to Gore; and U.S. Pat. No. 4,187,390 for Porous Products and Process Therefore issued Feb. 5, 1980 to Gore; the contents of each of which is incorporated herein by reference.

In an aspect of the invention, the steps of depositing the plurality of droplets on a surface (e.g., an exterior surface of a matrix of expanded fluoropolymer) and allowing the plurality of droplets to penetrate into the expanded polymer matrix and evaporating the non-aqueous liquid from the surface leaving a residue of nanoparticles may be conducted a plurality of times. According to an aspect of the invention, the process may deposit nanoparticles on a porous surface (e.g., an expanded material such as expanded polytetrafluoroethylene) such that the nanoparticles penetrate into the porous surface. More particularly, the process may deposit nanoparticles on a porous surface in such manner that the penetration of nanoparticles into the porous surface is controlled. This can be important in a variety of applications where nanoparticles are desired to be present at or near a surface (e.g., beneath a surface) but not penetrated entirely through or throughout a material.

The present invention encompasses the use of a silver nanoparticle sol composed of 25 to 5000 parts per million of silver nanoparticles; and 995000 to 999975 parts per million of a non-aqueous liquid to for the droplets that are deposited on the expanded polymer surface. For purposes of the present invention, a concentration of nanoparticles in non-aqueous characterized as 1,000 parts per million (i.e., 1,000 parts nanoparticles to 1,000,000 parts non-aqueous liquid) generally correspond to 1,000 micrograms (μg) of nanoparticles per 1,000,000 grams (g) of liquid which may be expressed as (m/g). In other words, a nanoparticle concentration of 1 part per million (i.e., 1 ppm) generally corresponds to a concentration of 1 μg/g for the types of nanoparticles and non-aqueous liquids employed in the present invention. Desirably, the silver nanoparticles have an effective diameter of less than 20 nm. The silver nanoparticle sol also has a viscosity of about 1 cP or less at 25° C. The non-aqueous liquid may be benzene, butanol, carbon tetrachloride, cyclohexane, 1,2-dichloroethane, dichloromethane, ethyl acetate, ethyl ether, iso-octane, methyl-t-butylether, methyl ethyl ketone, pentane, heptane, chloroform, toluene, and hexane and mixtures thereof.

The sol desirably has low viscosity and is adapted to forming droplets utilizing conventional droplet forming techniques. The sol is then processed to form a plurality of droplets utilizing conventional spray processes or techniques. For example, a spray process may utilize a centrifugal pressure nozzle, a solid cone nozzle, a fan spray nozzle, a sonic atomizer, a rotary atomizer, a flashing liquid jet, ultrasonic nozzles or combinations thereof. The spray process may utilize electrostatic charge.

These droplets are deposited on a surface (e.g., the exterior surface of the expanded polymer matrix) and allowed to penetrate. The penetration can be controlled by varying the rate at which the droplets are applied and the amount that is applied. Alternatively and/or additionally to forming droplets, it is contemplated that the process may deposit the sol on a surface by techniques selected from printing, dipping, brushing or combinations thereof. The surface to be treated may be a particular area, region, portion, or dimension of a medical device, device material, packaging material or combinations thereof. The surface may be hydrophobic or hydrophilic. The surface (or portions of the surface) may be pretreated to modify the surface energy to enhance the application of the sol or to help repel the sol. Non-polar non-aqueous liquids such as, for example, heptanes have been found to work particularly well on hydrophobic surfaces such as, for example, polytetrafluoroethylene. Without being held to a particular theory of operation, the surface energy of the nanoparticle sol formed from non-polar, non-aqueous liquid penetrates the matrix of expanded polymer to allow deposition of nanoparticles in the matrix. This is advantageous because the nanoparticles can adhere well to the nodes and fibrils of the matrix through van der Waals interaction, chemical interactions and/or mechanical interactions. Moreover, the presence of nanoparticles such as silver nanoparticles in the matrix adjacent the first exterior surface permits the elution of ions from the nanoparticles without the hindering effects of binders or other coatings and/or fixing agents that may impede the elution of ions. The elution of ions (e.g., silver ions) is important to provide antimicrobial properties to the composite material.

After the sol is deposited on the surface, the non-aqueous liquid is evaporated from the matrix to leave a residue of nanoparticles at or, more desirably, adjacent the surface of the matrix. A spray booth or similar structure with an exhaust system is useful to provide a flow of air to help evaporate the non-aqueous liquid and to properly handle the vapor. The residue of nanoparticles adheres to the surface of the article. The steps of depositing the sol (e.g., as a plurality of droplets or by other techniques) on a surface and evaporating the non-aqueous liquid from the surface leaving a residue of nanoparticles may be conducted a plurality of times.

The residue of nanoparticles may be designed to provide antimicrobial properties. Desirably, the nanoparticles are present at only the article surface. It is contemplated that the sol may further include other antimicrobial constituents including, but not limited to, copper nanoparticles, chlorohexidine, iodine, antibiotics and combinations thereof to enhance the antimicrobial properties of the residue.

The antimicrobial composite structure may be a particular area, region, portion, or dimension of a medical device, device material, packaging material or combinations thereof.

In one example, polytetrafluoroethylene material was treated selectively on the outer dimension of a tubular structure with nanoparticles of antimicrobial silver suspended in heptane, chloroform, and toluene, or mixtures thereof, by a spray technique utilizing a spray apparatus. In other examples, the nanoparticles have been applied to the surface of polytetrafluoroethylene material by dipping, brushing, or dripping the solvent/nanosilver mixture onto the surface of the material. Other examples represent additional materials that have been imparted with nanosilver in this fashion including silicone, paper, polyethylene, polystyrene, Styrofoam, polypropylene, wood, cotton, and polycarbonate. The nanosilver used in these examples is initially generated as an aqueous suspension according to commonly assigned U.S. Patent Application Publication No. 2007/0003603 for “Antimicrobial Silver Composition” published Jan. 4, 2007, the contents of which are incorporated herein by reference. U.S. Patent Application Publication No. 2007/0003603 corresponds to PCT/US2005/027261 and PCT International Application Publication WO2006026026A2). The silver nanoparticles generated in the aqueous suspension are then subjected to an extraction step that includes the total transfer of nanosilver from the aqueous phase into the organic phase of choice (e.g., heptane, chloroform and/or toluene).

### EXAMPLES

### Example 1

### Selective Spray Deposition on Polytetrafluoroethylene (PTFE)

It was desired to deposit nanosilver selectively to the outside diameter of a tubular structure. A spray deposition technique was developed to deposit silver in such a manner as to uniformly apply a coating on the outside of the tubular expanded PTFE or ePTFE (expanded polytetrafluoroethylene is available from W.L. Gore & Associates) material while leaving the inside diameter completely free of silver. The ePTFE graft material treated in this example was a hollow tube with an internal diameter of 6 mm and a length of up to 44 inches. The uniform application of the nanosilver was accomplished by rotating the tubular material on a mandrel that spans the length of the tubular structure. Referring to FIG. 2 of the drawings, there is shown a schematic drawing of an automated apparatus 10 for spraying the length of a tubular structure uniformly. The apparatus includes a base 12, a track 14 for a spray head 16 that can move along the track in the directions of the arrow “A” associated therewith. Parallel to the track 14 and in range of the spray head 16 is a mandrel 18 that is adapted to hold a tube or similar article. The mandrel 18 is configured to rotate. Rotation of speeds of between 500 and 4000 revolutions per minute (RPM) have been found to provide satisfactory results. The examples were produced at rotation speeds of about 3000 RPM.

This equipment could also utilize multi-axis motion control to precisely control the application of nanoparticles to complex substrate geometries. The nanoparticle sol may be contained in a reservoir 20. It is contemplated that the nanoparticle sol may be fed from an external reservoir. Features including a spray pass counter 22, motor controls 24, regulators for spray control, spray head position, and the like may be included.

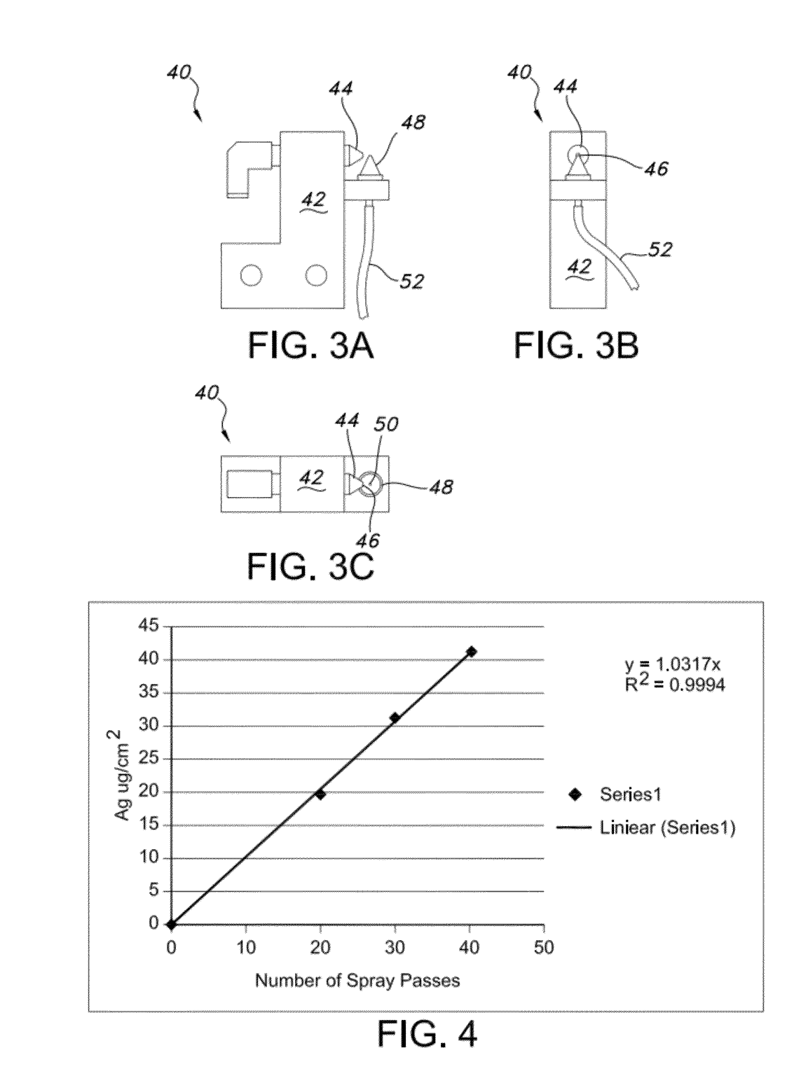

Referring to FIGS. 3A-C, there is shown an exemplary spray head utilized in the spray apparatus illustrated in FIG. 2. FIG. 3A is a side view of a modified Venturi spray head 40. More particularly, FIG. 3A is a view of the side of the spray head located on the left side when the spray head is viewed from the front. FIG. 3B is a front view of the modified Venturi spray head 40. More particularly, FIG. 3B is a view of the front face or front side of the spray head. FIG. 3C is a top view of the modified Venturi spray head 40. The spray head 40 includes mount 42 that supports a first housing 44 defining a first orifice 46 (referred to as an air or gas orifice 46—although gases such as, for example, nitrogen, carbon dioxide, argon or the like may be used instead of or in combination with air) for the supply of pressurized gas. The mount 42 of the spray head 40 also supports a second housing 48 defining a second orifice 50 (referred to as a Venturi orifice 50). A small diameter tube 52 is submerged into nanoparticle sol (not shown) in order to transfer the nanoparticle sol to the spray head 40 that sprays the mixture onto the intended substrate—which is desirably mounted on the mandrel 18. The Venturi orifice 50 is located in the path of the stream of gas exiting the gas orifice 46. Due to the pressure difference, the nanoparticle sol is drawn through the Venturi orifice 50 and into the moving gas flow exiting the gas orifice 46. The nanoparticle sol is projected as a fine spray of droplets onto the article mounted on the mandrel 18.

The spray coating was conducted in a specially designed and fabricated spray booth that included multi-axis spraying capabilities, specialized exhaust features to remove volatile organic vapors, and an automated programmable coating counter to control the number of spray coats and the point of shut-off for the spray head.

### Process:

This treatment process includes the following steps:
1. Formation of aqueous Ag nanoparticles (AgNP) mixture. This step involves the typical batching of a silver nanoparticle recipe (See U.S. Patent Application Publication No. 2007/0003603 for “Antimicrobial Silver Composition”). The preparation is summarized below:
   1 part by volume of ‘1×’ (16.67 g/L) Tween 20 surfactant (=Polysorbate 20 or polyoxyethylene (20) sorbitan monolaurate)
   1 part by volume 0.05M Sodium Acetate
   1 part by volume 0.15M Silver Nitrate
   Mixture is heated to ˜55 C.
   1/10 part by volume of N,N,N′,N′ tetramethylethylenediamine (TEMED).
   Mixture is maintained at ˜55 C. for 16+ hours.
2. Extraction of AgNP into Heptane to form AgNP:Heptane mixture. This step involves the destabilization of AgNP and re-dispersion into heptane.
   AgNP mixture is maintained at 55 C.
   Na Citrate is added to make the solution 2M (516 g/L). (A 7:3 volume ratio of AgNP:99% Isopropyl Alcohol (IPA) can also be used).
   Mixture is allowed to cool to room temperature under stirring. A brown to black oily precipitate will form.
   The aqueous layer is decanted, leaving behind the oily precipitate containing AgNP.
   An equal volume of heptane, chloroform, toluene, or mixtures thereof is added and stirred for up to 16 hours. The AgNP will re-disperse in this liquid, making it amber to brown in appearance.
   The organic layer is then decanted and filtered, leaving behind the oily precipitate.
   The concentration of this suspension can be monitored using UV/vis spectrophotometry at the 420 nm wavelength. A typical mixture will be diluted 1:3 with heptane and the absorbance at 420 nm recorded. The desired absorbance of this diluted mixture will be 1.5 AU. The Ag nanoparticles are thus suspended in heptane.
3. Treatment of ePTFE Material. This step involves the actual coating of the ePTFE material in the AgNP:Heptane mixture.
   The tubular ePTFE material is placed on provided stainless steel mandrels and stretched as completely as possible (i.e., without causing permanent deformation of or damage to the material). Stretching allows for a uniform coating of the ePTFE which is a very pliable and soft substrate. Without stretching the resulting coating is visually non-uniform. The mandrels must be dry and at no time are the mandrels or grafts to be handled with ungloved hands. The mandrels also prevent inadvertent spray treatment of the lumen of the tubular material with nanoparticles.
   The appropriate amount of AgNP:Heptane mixture is poured into a reservoir to supply the spray apparatus.
   The desired number of spray coatings is selected and the coating is performed.

After the ePTFE material was coated with silver, it was tested for antimicrobial efficacy utilizing a conventional 24 hour bacterial challenge assay. In such a test, the substrates are challenged with known bacterial count while immersed in medium for 24 hours. The medium was then appropriately diluted and plated on MHA (Mueller-Hinton Agar) plates to estimate the surviving bacterial count. A log reduction of bacteria exposed to the treated substrate over a 24-hour period is a typical test to measure antimicrobial activity. A reduction of 3-logs (99.9%) of bacteria is widely considered to indicate a coating or treatment that is highly effective as an antibacterial agent. Table A demonstrates the antimicrobial nature of the deposited nanosilver against Methicillin Resistant Staphylococcus Aureus (MRSA). In Table 1, T0 is the zero time inoculum and T1 is 24 hour time survivor count. The log T0 data is included to confirm that nothing was abnormally affecting bacterial growth on the untreated plates. The data in Table A below indicate a log reduction in excess of the 3-log threshold.

TABLE A
Demonstration of Antimicrobial Nanosilver Coating on PTFE against
MRSA

| | 24-Hour | | | | | |
|---|---|---|---|---|---|---|
| | | | | Untreated Control Substrate | | |
| | Silver Treated Substrate | | | (ID: AI 29507) | | |
| | (n = 3) | | | (n = 2) | | |
| | Log₁₀ | Log₁₀ | Log₁₀ | Log₁₀ | | Log₁₀ |
| Samples | T₀ | T1 | Reduction | T₀ | Log₁₀ T1 | Reduction |
| 0111-21A | 4.93 | 1.00 | 6.22 | 4.93 | 7.22 | — |
| 0111-21B | 4.84 | 2.48 | 4.78 | 4.84 | 7.26 | — |
| 0111-21C | 4.84 | 2.51 | 4.75 | 4.84 | 7.26 | — |

T₀: Zero time inoculum,
T1: 24-hour time survivor count
*Log reduction = Log₁₀ (Untreated Control Substrate at T1) − Log₁₀ (Treated Substrate at T1)

FIG. 4 illustrates the relative uniformity and predictability of results from the spray coating process described above in this Example 1. FIG. 4 is a graph of silver deposition expressed in units of micrograms per square centimeter on the y-axis and the number of spray passes on the x-axis. More particularly, the ePTFE tube was sprayed for approximately 20 seconds and was allowed to air dry for 30 seconds between each spray. The silver deposition was measured utilizing flame atomic absorption spectrophotometry (FAAS).

### Example 2

### Selective Nanosilver Deposition onto Paper and Other Materials by Brushing or Dripping

Paper of various constructions, including notebook paper, cardboard, particulates, was treated with nanosilver by dripping a mixture of an organic solvent and suspended nanoparticles onto a selected surface of material. This was conducted using chloroform, toluene, and heptane as the solvent or combinations thereof and nanosilver as the nanoparticles. The volatile nature of these solvents allows the solvent to evaporate before the untreated side of the substrate is saturated and therefore allows silver to be deposited only on one side of the paper. This method was also performed on materials made with polyethylene, polystyrene, Styrofoam (using only heptanes), polypropylene, wood, cotton (such as a gauze material), and polycarbonate. The advantage of solvent based nanosilver deposition is the rapid nature of the deposition time and the selectivity of the treatment method to render materials antimicrobial.

It will be recognized that the above methods and examples can be modified as appropriate without departing from the scope of the invention. The silver deposition step may be carried out at room temperature or optionally below or above room temperature. The substrate to be coated with nanosilver can undergo identical spray, dip, or brushing steps to increase the surface concentration of nanosilver as desired. Additionally, it has been verified that the AgNP:Organic mixture can be stored in excess of 6 months, the nanosilver particles remain uniformly suspended in the mixture, and the mixture remains viable for the coating process.

### Example 3

### Evaluation of Silver Nanoparticle Deposition on Expanded Polytetrafluoroethylene Tube

An exemplary antimicrobial composite structure in the form of the expanded polytetrafluoroethylene tubing treated with a silver nanoparticle sol according to the process described in Example 1 was prepared. The tubing was treated by twenty-five (25) spray passes. Measurements for expanded polytetrafluoroethylene tubing that was treated are as follows:

Outside Diameter: 0.78 cm

Inside Diameter: 0.60 cm

Sample Length: 15 cm

Outside Surface Area: 36.76 cm²

Density: 0.742 g/cc (theoretical=2.2 g/cc)

Percent Solid: 34% (based on density)

Percent Open: 66%

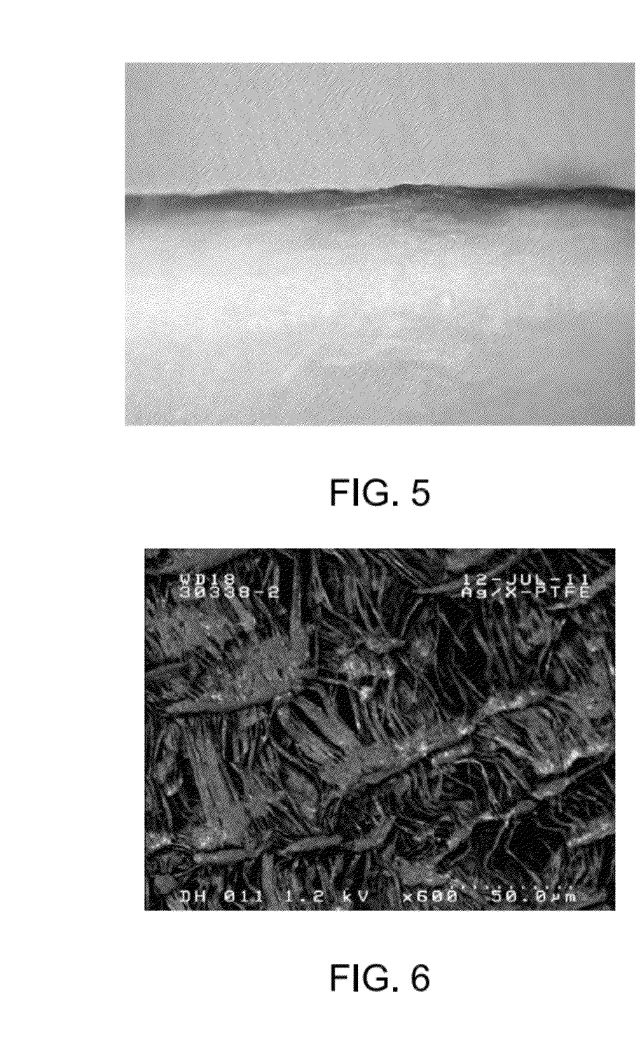

Referring to FIG. 5, there is shown a photographic image of an antimicrobial composite material in the form of a cross-section of the treated tubing. The image represents an approximately 400 micrometer cross-section of a treated tube. The darker treated region can be seen to be concentrated on a first exterior surface of the antimicrobial composite material (e.g., treated ePTFE tubing) with some penetration into an interior portion of the expanded polymer matrix. The treatment was applied via twenty-five (25) spray passes such that the treatment penetrated the first exterior surface to a depth of between about 10 to about 20 micrometers. Not shown in FIG. 5 is a second exterior surface from which silver nanoparticles were absent. In other words, FIG. 5 represents a matrix of fluoropolymer material, the matrix having a first exterior surface expanded to fibrils and nodes, a second exterior surface and an interior portion between the exterior surfaces; and nanoparticles present in the interior portion adjacent the first exterior surface but not in the interior portion adjacent the second exterior surface.

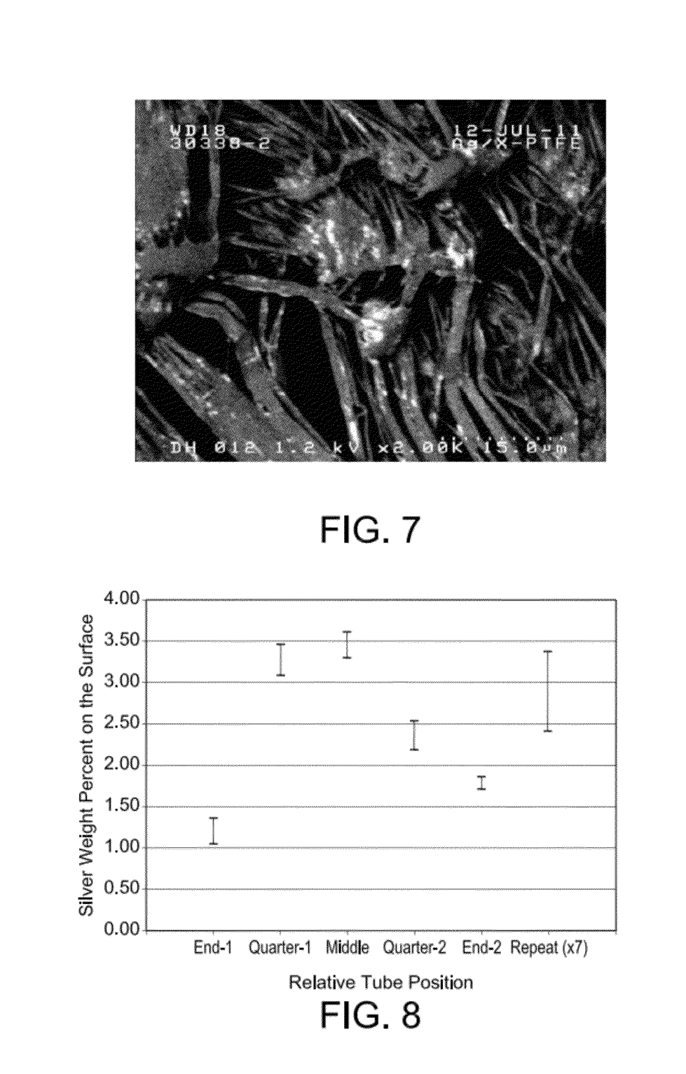

FIG. 6 is a scanning electron photomicrograph of a portion of an exemplary expanded polymer matrix of FIG. 5 at a linear magnification of 200×. As can be seen in the photomicrograph, the node and fibril microstructure of the expanded polytetrafluoroethylene is visible. FIG. 7 is a scanning electron photomicrograph of a portion of the expanded polymer matrix at a linear magnification of 2000×. As can be seen in the photomicrograph, numerous fibrils, portions of nodes and some evidence of deposited silver nanoparticles are visible.

Two separate sets of tubing sections were analyzed. First, a section of tubing approximately one-half inch (˜13 mm) long was cut from approximately the center region of the tubing. This section was mounted and a series of seven (7) energy dispersive x-ray spectroscopy (EDS) analyses were performed across the length of the sample. Secondly, five sections (approximately 13 mm long) were cut one from: each end (i.e., approximately 1 inch inward towards the center from the end), each quarter point (i.e., approximately equidistant from the “end” sample and the mid-point) and the one mid-point. These samples had the EDS analysis performed at two points. The calculated silver weight percent for the high and low points for the five sections taken from along the length of the tube along with the high (average plus 1-standard deviation) and the low (average minus 1-standard deviation) of the multiple analyses on one piece are shown in FIG. 8 in the form of a graph in which the y-axis is the amount of silver detected at or adjacent the surface of the sample (expressed in terms of weight percent of the sample) and in which the x-axis represents samples taken at various positions across the length of an antimicrobial composite matrix (i.e., in this case, the treated tube) namely, approximately one inch inward towards the center from the first end (End-1), the first quarter (Quarter-1) which is the location equidistant between the first “end” sample and the mid-point, the approximate mid-point (Middle), the second quarter (Quarter-2) which is the location equidistant between the second “end” sample and the mid-point, and approximately one inch inward towards the center from the second end (End-2). At the far right of the x-axis is a graphical representation of the amount of silver from the series of seven (7) energy dispersive x-ray spectroscopy (EDS) analyses of the separate section of tubing approximately one-half inch (−13 mm) long that was cut from approximately the center region of the tubing.

From FIG. 8, it can be seen that the silver concentration ranges from about 1.0 to 3.6 weight percent Ag. The silver concentration appears to be greatest near the middle (in terms of the length dimension) of the tubing. The small range ‘scatter’ (from the series of seven (7) repeat EDS analyses) is greater than that seen on the individual sections.

The overall trend that the end regions (both taken approximately 1 inch in from the end) have a lower silver intensity than the middle region is most likely a valid comparison. The size of the spread from the seven analyses that were performed along the same ˜13 mm piece suggests that the surface is far from ideal for quantitative analysis. From FIGS. 6 and 7 it can be seen that the polymeric microporous material in the form of the matrix of expanded polytetrafluoroethylene is made from layers of overlapping strands that can hold the silver. This rough, multi-dimensional surface is non-particularly well suited for an analysis collecting information from the very top 1 micrometer of the surface.

Various measurements of two samples of expanded polytetrafluoroethylene tubing treated utilizing the spray process described in Example 1 are reported in Table B below. These measurements include dimensions, weights and silver concentrations based on EDS analyses.

TABLE B
Measurements For The Expanded PTFE Tube

| | | | Outside | Ag Conc | | | |
|---|---|---|---|---|---|---|---|
| Sample | | Weight | surface area | (ppm) (ug | Ag Conc | Ug Ag/cm² | ug Ag/cm |
| ID | L (cm) | (g) | per g (cm²/g) | Ag/g) | (ug) | (per area) | (per length) |
| X-1 | 15 | 2.19775 | 16.726 | 215 | 472.5 | 12.86 | 31.8 |
| X-2 | 15 | 2.10654 | 17.450 | 230 | 484.5 | 13.18 | 32.3 |

Surface Analysis: X-ray photoelectron spectroscopy (XPS) was used to examine the chemistry on the very outer (10 nanometer) surface of the tubing. A section of tubing from near the middle of the sample was cut out and mounted for analysis. This sample had two definitive shades (darker and lighter) and each region had three areas analyzed. In a representative XPS wide scan from the treated tubing, the dominance of fluorine is seen for these analyses just like for the EDS. Table C shows the averages of the XPS analyses for the two regions.

TABLE C
XPS Results for the Silver Treated Tubing

| | Atomic Percent | | | | |
|---|---|---|---|---|---|
| Sample | C | O | F | Si | Ag |
| Darker Area | 35.3 | 2.0 | 61.6 | 0.7 | 0.4 |
| Lighter Area | 34.2 | 1.2 | 64.3 | 0.0 | 0.3 |

These results show that the surface is predominantly that of the PTFE (i.e., CF₂) and that the silver is found at trace levels (<1 at %) for both the darker and lighter area.

The main difference between the darker and lighter regions is the darker region has slightly more carbon, oxygen, and silicon and less fluorine than the lighter region. But it should be noted that like the EDS analysis the surface roughness will also influence the XPS results.

From a representative XPS spectrum, the carbon is represented by two peaks. The smaller peak at 284.6 eV is from aliphatic carbon (—C—H) that is most likely from residual carbonaceous material deposited during the treatment. The larger carbon peak at ˜292 eV is from the carbon that is bound to two fluorine atoms in the PTFE matrix (—CF₂). Curve fitting these peaks allows the percent area of the two carbon peaks to be determined. Using the area percent and the total atomic percent for carbon the atomic percent carbon for the two functionalities can be determined. Table D shows the curve fitting results for the carbon peaks.

TABLE D
Carbon 1s Curve Fitting for the Silver Treated Tubing

| | |
|---|---|
| Carbon Area | Carbon Atomic |
| Percent | Percent |

| Sample | C—H | CF₂ | C—H | CF₂ |
|---|---|---|---|---|
| Darker Area | 24.6 | 75.4 | 8.7 | 26.6 |
| Lighter Area | 15.9 | 84.1 | 5.4 | 28.8 |

From this it can be seen that the aliphatic carbon is the minor component and that the darker area has approximately 1.6 times more aliphatic carbon than the lighter area. Overall, a very light coating of surface oriented silver treatment was found along the length of the tubing with the highest levels being found near the midpoint of the sample. When these results (relatively low amount of silver in the first 10 nanometers of the surface) are considered in combination with the overall weight percentage of silver, it is clear that silver nanoparticles are distributed in the expanded polymer matrix in the interior portion adjacent the first exterior surface.

### Example 4

### Evaluation of Silver Nanoparticle Deposition on Polytetrafluoroethylene Film

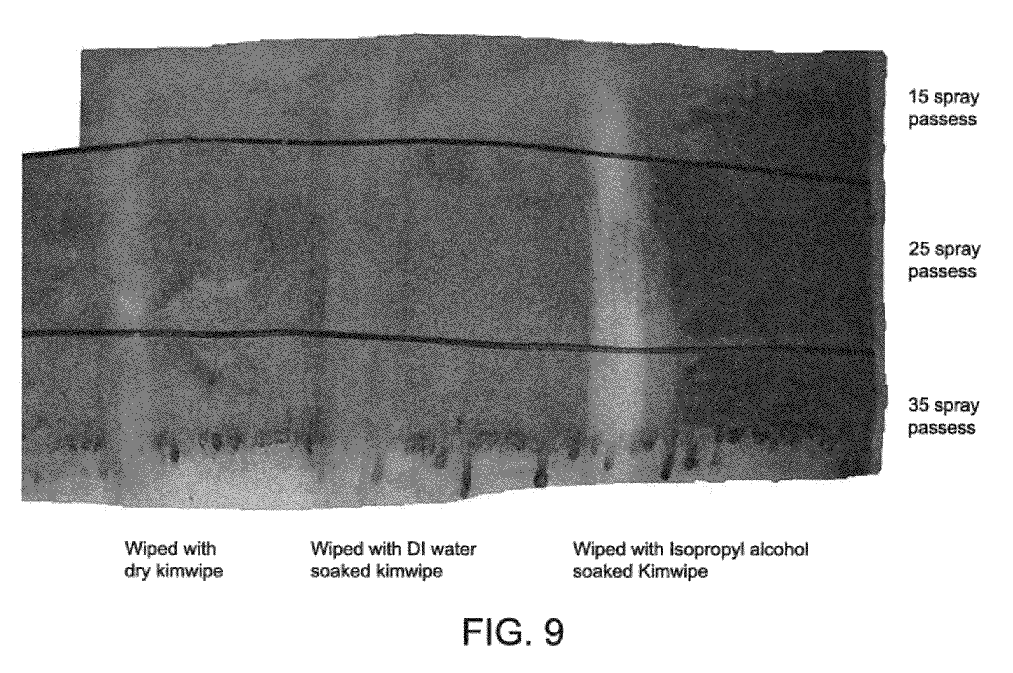

A length of Teflon® polytetrafluoroethylene film approximately 0.005 inch thick and 12 inches wide and having a smooth finish (McMaster-Carr, Part No. 8569K38) was sprayed horizontally with the silver nanoparticle sol (silver nanoparticles in heptane) prepared according to Example 1. Three zones were created by varying the number of treatment passes. Referring to FIG. 9, there is shown a photograph (backlit) of the sheet highlighting the treated portion is approximately 28.5 cm×9.6 cm in size. The film was sprayed horizontally with the silver nanoparticle sol along the long axis of the film. The three zones are as follows: 15 passes (top); 25 passes (middle); and 30 passes (bottom).

After bring allowed to dry, the sample was subjected to a single wipe pass utilizing a KimTech Science™ KimWipes®—delicate task wiper, available from Kimberly-Clark Corporation, at three separate locations to assess the durability of the deposited silver nanoparticle treatment on the film. The wipe was drawn across the sample using the index finger of the tester while apply moderate pressure (about equal to the force typically used to write with a pencil or other writing instrument on paper). One wipe was used dry, one wipe was saturated with deionized water and one wiper was saturated with an aqueous solution of isopropyl alcohol (approximately 70% isopropyl alcohol and approximately 30% water).

As can be seen in FIG. 9, the dry wipe (“dry wipe”) test area at the left side of the photograph shows in a manner that is detectable with the unaided eye that dark silver treatment has been at least partially removed by wiping. The deionized water wipe (“DI wipe”) test area at the center of the photograph shows in a manner that is detectable with the unaided eye that dark silver treatment has been at least partially removed by wiping. The isopropyl alcohol wipe (“IPA wipe”) test area at the right side of the photograph shows in a manner that is detectable with the unaided eye that dark silver treatment has been at least partially if not substantially removed by wiping. In each case, removal of the deposited silver nanoparticle treatment is visible and the isopropyl alcohol wipe appears to have removed the most treatment.

As noted above, the silver nanoparticle sol was applied to create three different treatments zones by varying the number a treatment passes: 15 passes (top); 25 passes (middle); and 30 passes (bottom). A sample from each zone and a control (i.e., untreated Teflon® polytetrafluoroethylene film) was subjected to energy dispersive x-ray spectroscopy (EDS) analyses. The results are reported in Table E below.

TABLE E
EDS Analysis of Film Samples

| | Weight Percent | | |
|---|---|---|---|
| Sample | C | F | Ag |
| 15x | 19.9 | 76.7 | 3.3 |
| 25x | 21.8 | 72.7 | 5.5 |
| 30x | 23.1 | 74.0 | 3.0 |
| control | 24.0 | 76.0 | — |

Even though the samples are carbon coated, the C and F values are consistent with that expected for the Teflon substrate since the carbon coating is extremely thin.

The 15× and 30× regions have similar silver loadings with the 25× region having the highest silver level.

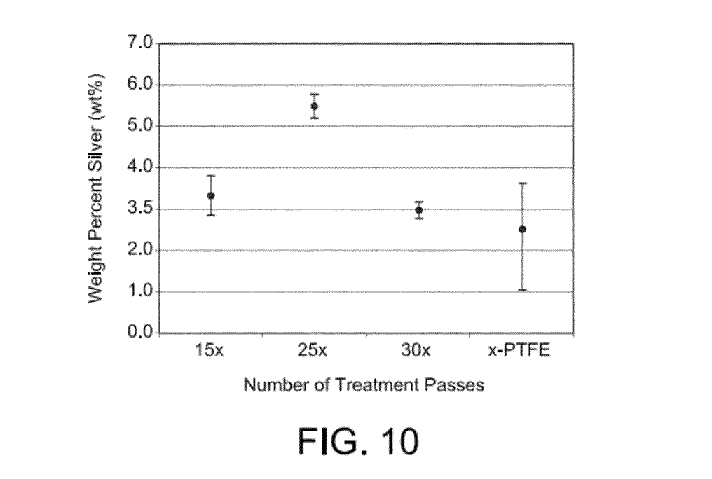

Referring now to FIG. 10 of the drawings, there is shown an illustration of a graph of the amount of silver nanoparticles from the comparative polytetrafluoroethylene film material described above and from the antimicrobial composite material (e.g., an expanded polytetrafluoroethylene tube) of Example 3. The amount of detected silver is expressed as a weight percentage on the y-axis and the number of treatment passes (identified as 15×, 25×, and 30×) of the comparative film material is on the x-axis, except for the antimicrobial composite material (identified as “x-PTFE”)—which illustrates a summary of the range of weight percentages shown in FIG. 8—that is, average and high-low range of values obtained for the expanded polytetrafluoroethylene tube. The upper half of the x-PTFE tube range is consistent with the film samples (15×, 30×) but the lower half is significantly lower—this is mainly a function of the surface roughness for the x-PTFE tube sample.

A 48 mm diameter circle of the untreated film (0.005″ thick) was cut and measured. The measurements are as follows:

Weight: 0.5261+/−0.0017 g

Area: 18.096 cm²

Area per unit weight: 34.398 cm²/g

Density: 2.29 g/cc (theoretical=2.2 g/cc)

Measurements from samples of smooth polytetrafluoroethylene film treated utilizing the above-described spray process are reported in Table F below. These measurements reflect two samples per zone from each of the three separate zones created by varying the number of spray passes. Because the amount of added silver is in microgram quantities, the weight per unit area for the treated film samples was assumed to be the same as for the untreated film for sample having an area on the order of 10 cm² or so. These measurements include dimensions, weights and silver concentrations based on EDS analyses.

TABLE F
Measurements For The Treated Film (15, 25, and 30 passes)

| | | | | Ag Conc | Ag | | Average | Avg ug |
|---|---|---|---|---|---|---|---|---|
| Sample | Avg size | Weight | Area (by | (ppm) (ug | Conc | ug | ug | Ag/cm² per |
| ID | (cm²) | (g) | wt.) (cm²) | Ag/g) | (ug) | Ag/cm² | Ag/cm² | pass |
| 15-1 | 2.2 × 5.4 | 0.34517 | 11.87 | 132 | 45.56 | 3.84 | | |
| 15-2 | 2.4 × 5.0 | 0.33763 | 11.61 | 176 | 59.42 | 5.12 | 4.48 | 0.298 |
| 25-1 | 2.3 × 5.3 | 0.35081 | 12.07 | 287 | 100.68 | 8.34 | | |
| 25-2 | 2.3 × 4.9 | 0.31998 | 11.01 | 223 | 71.36 | 6.48 | 7.41 | 0.297 |
| 30-1 | 2.1 × 5.2 | 0.32012 | 11.01 | 316 | 101.16 | 9.19 | | |
| 30-2 | 2.0 × 5.7 | 0.30590 | 10.52 | 300 | 91.77 | 8.72 | 8.95 | 0.298 |

The quantitative values averaged over a larger area still show some variation within each zone, but on average the amount of silver added per pass (application) is remarkably the same.

X-ray photoelectron spectroscopy (XPS) was used to examine the chemistry on the very outer (10 nanometer) surface of the film samples from each of the three separate areas of treatment (i.e., 15×, 25× and 30×). Table G shows the results of the XPS analyses for the three areas and for the antimicrobial composite structure (e.g., expanded polytetrafluoroethylene tube).

TABLE G
Surface Analysis by XPS of the Treated Film and x-PTFE Tube

| | Atomic Percent | | | | | | |
|---|---|---|---|---|---|---|---|
| Sample | C | O | F | Na | Si | Ag | % exposed PTFE |
| Film 15x | 48.4 | 4.9 | 43.1 | 0.3 | 0.1 | 3.4 | 64.7 |
| Film 25x | 49.1 | 5.8 | 40.4 | 0.4 | 1.1 | 3.7 | 60.6 |
| Film 30x | 45.2 | 4.6 | 46.7 | 0.2 | 0.4 | 3.2 | 70.0 |
| X-PTFE | 34.8 | 1.6 | 63.0 | — | 0.4 | 0.3 | 94.4 |

The polytetrafluoroethylene film is a solid surface substrate that essentially is coated as a two-dimensional structure. The surface analysis shows that more of the polytetrafluoroethylene surface is covered for the film as compared to the expanded polytetrafluoroethylene microstructure of the tube and this also corresponds into a higher silver concentration for the solid surface as compared to the outermost layer of the expanded polytetrafluoroethylene microstructure of the tube.

Generally speaking, the quantitative data suggests that the expanded polytetrafluoroethylene microstructure of the tube is able to retain (accept) more silver per unit area than the solid polytetrafluoroethylene film. This highlights an advantage of the antimicrobial composite material in that more nanoparticle material is able to be captured by the microstructure.

The EDS analysis suggests that the outer 1 micrometer of the expanded polytetrafluoroethylene microstructure of the tube (i.e., expanded PTFE tube) has slightly less silver than the polytetrafluoroethylene film's surface (i.e., the Teflon® Film). The

XPS analysis suggests that the outer 10 nanometers of the expanded polytetrafluoroethylene microstructure of the tube has significantly less silver than the corresponding portion of the polytetrafluoroethylene film's outer surface. This leads to a silver analysis gradient as follows:

Outer Surface (10 nanometers): Teflon® Film>expanded PTFE tube

Near Surface (1 micrometer): Teflon® Film≧expanded PTFE tube

Bulk: expanded PTFE tube>Teflon® Film

While the inventors should not be held to a particular theory of operation, this gradient analysis shows that the open structure or microstructure of the expanded PTFE tube allows for a three dimensional coating (albeit thin) that contains more silver per unit area than is achieved for the solid polytetrafluoroethylene film even for equivalent treatments (25 passes for the expanded polytetrafluoroethylene tubing and 25-30 passes for the polytetrafluoroethylene film). It should be noted that the polytetrafluoroethylene film treated with 15 spray passes had even less silver per unit area than for the expanded polytetrafluoroethylene tubing treated with 25 spray passes.

Various scanning electron microscopy images (SEM images) of the film were analyzed. These images suggest that, for the first 15 spray passes, the deposition of the silver is mainly influenced by the morphology (roughness) of the film. For higher numbers of spray passes (i.e., 25-30 spray passes) the deposition takes on the appearance of more closely resembling overlapping droplets with outer rim particulate deposition (i.e., build up at the outer rim of the droplet). Since the particles on the film's surface do not have anywhere to go, the build-up/pattern of the deposit becomes influenced by the addition of more spray treatment (i.e., more application of the silver nanoparticle sol).

### Experimental Methods

SEM: Scanning electron microscopy (SEM) was performed with a Hitachi S4500 field emission scanning electron microscope (FESEM) operating at 1.2 kV. The samples were dusted onto conductive carbon tape and imaged without coating. Digital images were collected using Quartz PCI software.

EDS: Energy dispersive x-ray spectroscopy was performed using an Oxford Instruments Pentafet Si(Li) solid state EDS detector with Link™ software. A collection voltage of 20 kV was used and the samples were gold coated to mitigate charging.

XPS: Surface analysis was performed by x-ray photoelectron spectroscopy (XPS) using a Fisons M-Probe spectrometer equipped with monochromatic Al Kα x-rays. Atomic sensitivity factors, supplied with the Fisons M-Probe spectrometer, were used to establish the relative atomic concentration of the elements detected by the spectrometer. Spot size of ˜1 mm was used. Charge neutralization was accomplished using the electron flood gun/screen (FGS) method. Wide scans were performed to document the elemental composition of the surface.

While various patents have been incorporated herein by reference, to the extent there is any inconsistency between incorporated material and that of the written specification, the written specification shall control. In addition, while the disclosure has been described in detail with respect to specific embodiments thereof, it will be apparent to those skilled in the art that various alterations, modifications and other changes may be made to the disclosure without departing from the spirit and scope of the present disclosure. It is therefore intended that the claims cover all such modifications, alterations and other changes encompassed by the appended claims.

We claim:

1. An antimicrobial composite structure comprising:
a microporous polymeric material, the material having a first exterior surface, a second exterior surface and an interior portion between the exterior surfaces; and
nanoparticles present in interstices adjacent the first exterior surface but not in interstices adjacent the second exterior surface.

2. The composite structure of claim 1, wherein the nanoparticles resist removal by wiping.

3. The composite structure of claim 1, wherein the nanoparticles are silver nanoparticles.

4. The composite structure of claim 1, wherein the first exterior surface comprises nodes and fibrils of expanded fluoropolymer material.

5. The composite structure of claim 4, wherein the expanded fluoropolymer material is expanded polytetrafluoroethylene.

6. The composite structure of claim 1, wherein the composite structure comprises an area, region, portion, or dimension of a medical device, device material, packaging material or combinations thereof.

7. The composite structure of claim 1, wherein the nanoparticles are present in the interstices to a depth of from about 5 to about 20 micrometers from the first surface.

8. The composite structure of claim 1, further comprising copper nanoparticles, chlorohexidine, iodine, antibiotics and combinations thereof.

9. An antimicrobial composite structure comprising:
a polymeric material, the materials having a first exterior surface expanded to fibrils and nodes, a second exterior surface and an interior portion between the exterior surfaces; and
nanoparticles present in the interior portion adjacent the first exterior surface but not in the interior portion adjacent the second exterior surface.

10. The composite structure of claim 9, wherein the nanoparticles resist removal by wiping.

11. The composite structure of claim 9, wherein the nanoparticles are silver nanoparticles.

12. The composite structure of claim 9, wherein the polymeric material is expanded polytetrafluoroethylene.

13. The composite structure of claim 9, wherein the composite structure comprises a particular area, region, portion, or dimension of a medical device, device material, packaging material or combinations thereof.

14. The composite structure of claim 9, wherein the nanoparticles are present in the interstices to a depth of from about 5 to about 20 micrometers from the first exterior surface.

15. The composite structure of claim 9, further comprising copper nanoparticles, chlorohexidine, iodine, antibiotics and combinations thereof.

16. An antimicrobial composite structure comprising:
a matrix of polytetrafluoroethylene material, the matrix having an exterior surface expanded to fibrils and nodes; and
silver nanoparticles present in the matrix adjacent only the exterior surface but not completely throughout the matrix, the nanoparticles having resistance to removal from the matrix by wiping.

17. The composite structure of claim 16, wherein the composite structure comprises a particular area, region, portion, or dimension of a medical device, device material, packaging material or combinations thereof.

18. The composite structure of claim 16, wherein the nanoparticles are present in the matrix to a depth of from about 5 to about 20 micrometers from the exterior surface.

19. The composite structure of claim 16, further comprising copper nanoparticles, chlorohexidine, iodine, antibiotics and combinations thereof.
